(19) United States
(12) Patent Application Publication
(10) Pub. No.: US 2017/0296558 A1
(43) Pub. Date: Oct. 19, 2017

(54) USE OF 2AMD AND 2MD TO TREAT FIBROSIS

(71) Applicant: WISCONSIN ALUMNI RESEARCH FOUNDATION, Madison, WI (US)

(72) Inventors: Hector F. DeLuca, Deerfield, WI (US); Hans W. Sollinger, Madison, WI (US); Bianca Raquel Tomasini-Johansson, Madison, WI (US); Lori A. Plum, Arena, WI (US); Debra Hullett, Oregon, WI (US)

(21) Appl. No.: 15/450,510

(22) Filed: Mar. 6, 2017

Related U.S. Application Data
(60) Provisional application No. 62/323,938, filed on Apr. 18, 2016.

(51) Int. Cl. A61K 31/593; A61K 9/00; A61K 38/13

(52) U.S. Cl. CPC A61K 31/593; A61K 38/13; A61K 9/0019; A61K 9/0053

(57) ABSTRACT

The invention relates to using 2-methylene-19-nor-(20S)-1α,25-(OH)₂D₃ (2MD) or 2α-methyl-19-nor-(20S)-1α,25-(OH)₂D₃ (2AMD) to prevent nephrotoxicity and/or fibrosis in a patient, preferably without causing hypercalcemia in the patient.

### CROSS REFERENCE TO RELATED APPLICATIONS

This application claims the benefit of U.S. Provisional Patent Application 62/323,938 filed Apr. 18, 2016, which is incorporated by reference herein.

### STATEMENT REGARDING FEDERALLY SPONSORED RESEARCH/DEVELOPMENT

N/A

### BACKGROUND

Fibrosis has been estimated to be a contributing factor of up to 45% of deaths world-wide. Fibrotic disease can occur in a vast majority of tissues and organs throughout the body and can include a wide variety of diseases, such as idiopathic pulmonary fibrosis, renal fibrosis, liver fibrosis, diabetic nephropathy, and scleroderma.

Vitamin D analogs have been used in multiple clinical trials involving chronic kidney disease, hemodialysis, and hyper-parathyroidism. Ongoing trials testing the effects of vitamin D supplementation on patients undergoing kidney transplantation utilize cholecalciferol [1], which is known to have hypercalcemic effects with deleterious outcomes on patients' cardiovascular systems.

Novel selective analogs of vitamin D are known to be hyperactive Vitamin D receptor agonists at doses that do not elicit hypercalcemia [2, 3].

In the present invention described below, two such analogs, 2AMD (2α-methyl-19-nor-(20S)-1α,25-dihydroxyvitamin D₃) and 2MD (2-methylene-19-nor-(20S)-1α,25-dihydroxivitamin D₃) are studied for their ability to protect kidneys from cyclosporine (CsA) induced nephrotoxicity. This model is relevant to transplantation as CsA is an immunosuppressant given to reduce organ rejection but which eventually promotes renal vasoconstriction ending in epithelial cell apoptosis and interstitial fibrosis [4]. As described below, various doses of 2AMD were tested to identify the nephroprotective dose which would not raise serum calcium levels and found that 2AMD at 5 ng/kg rat body weight was optimal in nephroprotection. In addition, the present invention shows that 2AMD is effective in preventing renal fibrosis in an acute renal injury model, the unilateral ureteral obstruction model (UUO).

### DESCRIPTION OF DRAWINGS

The patent or application file contains at least one drawing in color. Copies of this patent or patent application with color drawings will be provided by the Office upon request and payment of the necessary fee.

FIG. 1. Serum calcium levels for rats dosed with CsA and various vitamin D analogs. Mean, SD

FIG. 2A. Representative kidney sections from paraffin sections stained with H&E.

FIG. 2B. Quantization of eosin (pink hue associated with native kidneys as in Cremo-PEG) of 6 random images from the central cortical area of each rat kidney was performed.

FIG. 3. Kidney sections were stained with picrosirius red representing mostly collagens I and III, thereby the fibrotic score for the tissues. Quantization of birefringence elicited with polarized light was performed for 6 random images in the central cortical of each kidney using Image J. Mean, SD

FIG. 4. Weight of animals at harvest following 28 days of CsA+/−Vitamin D analog treatments. Mean, SD.

FIG. 5. Serum tests for kidney function. BUN and creatinine were measured using an automated chemistry analyzer (VETTEST). Mean, SD

FIG. 6. Urinary protein/creatinine ratio. Protein and creatinine ratio was obtained from individual measurement of protein and creatinine using an automated chemistry analyzer (VETTEST). Mean, SD

FIG. 7. Effect of 2AMD on collagen in UUO kidney sections. Graphs depict quantization of birefringence from picrosirius staining of UUO-treated kidneys from rats given 2.5 ng/kg (1×) or 5 ng/kg (2×) 2AMD or equivalent vehicle volumes. Daily oral administration of 2AMD was started 7 days prior to UUO and continued for 7 days (left panel) or given for 14 days starting on the day of UUO.

### SUMMARY OF THE INVENTION

In one embodiment, the present invention is a method of treating fibrosis or symptoms thereof in a patient, the method comprising administering a therapeutically effective amount of a compound selected from the group consisting of 2-methylene-19-nor-(20S)-1α,25-(OH)₂D₃ (2MD) and 2α-methyl-19-nor-(20S)-1α,25-(OH)₂D₃ (2AMD), or pharmaceutically acceptable salts thereof, to the patient. Preferably, hypercalcemia is not induced in the patient during treatment.

In one embodiment, the compound is formulated in a formulation selected from the group consisting of an oral, topical, transdermal, parenteral, injection, and infusion dosage form.

In one embodiment, the fibrosis is a result of treatment with a calcineurin inhibitor (CNI), preferably cyclosporine (CsA).

In one embodiment, the fibrosis is a result of acute renal injury.

In one embodiment, the patient is a mammal, preferably a human.

### DESCRIPTION OF THE INVENTION

Disclosed are methods of treating and/or preventing fibrosis or symptoms thereof. Also disclosed are methods of treating and/or preventing immunosuppressant induced nephrotoxicity or symptoms thereof.

Unless defined otherwise, all technical and scientific terms used herein have the same meanings as commonly understood by one of ordinary skill in the art to which this invention belongs.

In the specification and in the claims, the terms “including” and “comprising” are open-ended terms and should be interpreted to mean “including, but not limited to.” These terms encompass the more restrictive terms “consisting essentially of” and “consisting of” It is also to be noted that the terms “comprising,” “including,” “characterized by” and “having” can be used interchangeably.

As used herein and in the appended claims, the singular forms “a,” “an,” and “the” include plural reference unless the context clearly dictates otherwise. As well, the terms “a” (or “an”), “one or more” and “at least one” can be used interchangeably herein.

Where a range of values is provided, it is understood that each intervening value, and any combination or sub-combination of intervening values, between the upper and lower limit of that range and any other stated or intervening value in that stated range, is encompassed within the range of values recited.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number, and thus will typically refer to a number or value that is 10% below or above the specifically recited number or value.

The disclosed methods may be utilized to treat and/or prevent fibrosis in a patient in need thereof. A patient in need thereof may include, but is not limited to, a patient having or at risk for developing fibrosis. A patient in need thereof may include, but is not limited to, patients suffering from idiopathic pulmonary fibrosis, COPD, cystic fibrosis, liver fibrosis, cirrhosis, diabetic nephropathy, renal fibrosis , nephrogenic system fibrosis, atrial fibrosis, endomyocardial fibrosis, cardiovascular remodeling, arthrofibrosis, myelofibrosis, Chron's disease, scleroderma, idiopathic retroperitoneal fibrosis, pancreatic fibrosis, IgG4-related disease, graft versus host disease, rejection of transplanted organ, glaucoma, post-traumatic or post-burn fibrotic changes such as scar formation which restrict movements or cause cosmetic problems, and some cancers, including but not limited to breast and pancreatic. Fibrosis may be diagnosis or detected in a patient by hematoxylin and eosin staining, picrosirius red staining, or trichrome staining. A patient in need thereof may be identified by increased levels of collagen as identified by hematoxylin and eosin staining, picrosirius red staining, or trichrome staining A patient in need thereof may also be a patient with indicators for increased tissue collagen levels as an indicator of fibrosis.

The disclosed methods may be utilized to treat and/or prevent immunosuppressant induced nephrotoxicity in a patient in need thereof. A patient in need thereof may include, but is not limited to, a patient taking an immunosuppressant agent to prevent organ rejection following a transplant. A patient in need thereof may include, but is not limited to, a patient who will be undergoing a transplant and is soon to be taking an immunosuppressive agent.

By “fibrosis” we mean the formation and accumulation of excess fibrous connective tissue, such as collagen, in an organ or tissue. Fibrosis may be a reparative feature of the organ or tissue critical to healing, such as scar formation, or it may be a reactive and pathological state of excess fibrous tissue deposition into an organ or tissue which disrupts the structure and function of the underlying organ or tissue. Fibrosis may include any one of a number of fibrotic diseases, including but not limited to idiopathic pulmonary fibrosis, COPD, cystic fibrosis, liver fibrosis, cirrhosis, diabetic nephropathy, renal fibrosis , nephrogenic system fibrosis, atrial fibrosis, endomyocardial fibrosis, cardiovascular remodeling, arthrofibrosis, myelofibrosis, Crohn's disease, scleroderma, idiopathic retroperitoneal fibrosis, pancreatic fibrosis, IgG4-related disease, graft versus host disease, rejection of transplanted organ, glaucoma, post-traumatic or post-burn fibrotic changes such as scar formation which restrict movements or cause cosmetic problems, and some cancers, including but not limited to breast and pancreatic.

Fibrosis can be induced or caused by any number of conditions, including but not limited to genetic conditions, various types of cancers, inflammation, chronic wounds, chronic antibody-mediated rejection, injury, certain medications, infections, metabolic disease, autoimmune diseases, vasoconstriction and subsequent ischemia. Fibrosis caused by any of these conditions is within the scope of the present invention.

In addition, fibrosis can also be idiopathic or a symptom or side effect of a drug treatment, chemotherapy treatment, palliative care, immunosuppressive treatment, prophylactic treatment or medical protocol used to treat an unrelated underlying condition or in conjunction with any number of surgical, medical or orthopedic treatments or therapies. In one embodiment, fibrosis is caused by the toxicity of an immunosuppressive agent used in conjunction with an organ transplant procedure. In one embodiment, the immunosuppressive agent is a calcineurin inhibitor (CNI). In one embodiment the CNI is cyclosporine (CsA).

By “immunosuppressive agent” we mean a drug that inhibits or prevents activity of the immune system. Immunosuppressive agents are used in immunosuppressive therapies to prevent rejection of transplanted organs and tissues, treat autoimmune diseases, and treat other non-autoimmune inflammatory diseases. Immunosuppressive agents may include, but are not limited to, glucocorticoids, cytostatics, antibodies, calcineurin inhibitors, interferons, opioids, TNF binding proteins, mycophenolates, and small biological agents. A preferred immunosuppressive agent is a CNI.

By “calcineurin inhibitor” (CNI) we mean an inhibitor of calcineurin, a calcium- and calmodulin-dependent phosphatase. CNIs are immunosuppressive agents used in a number of treatments including but not limited to, preventing graft rejection in organ transplant patients and treatment of rheumatoid arthritis, psoriasis, skin rash, eczema, and atopic dermatitis. CNIs include, but are not limited to, cyclosporine (CsA) and tacrolimus. CNIs can promote renal vasoconstriction leading to hypoxia damaging kidney tubules resulting in epithelial cell death and kidney malfunction. CNIs can also directly trigger the release of profibrotic cytokines, including but not limited to Transforming Growth Factor-β (TGF-β) and Monocyte Chemoattractant Protein-1 (MCP-1), which promote the excessive secretion and deposition of extracellular matrix proteins including collagen and fibronectin.

By “nephrotoxicity” we mean a poisonous side effect of chemicals, drugs or medication on the kidneys. Nephrotoxicity can be characterized by increased levels of blood urea nitrogen (BUN), creatinine, and an increased urinary protein/creatinine ratio. Nephrotoxicity can be caused by a number of chemicals and drugs including, but not limited to, immunosuppressive agents such as CNIs.

### Compounds of the Present Invention

2-methylene-19-nor-(20S)-1α,25-dihydroxyvitaminD3 (2MD) is an analog of 1,25(OH)₂D₃ which has been shown to have increased in vivo potency toward bone but not on intestinal calcium absorption. The overall synthesis of 2MD is illustrated and described more completely in U.S. Pat. No. 5,843,928, issued Dec. 1, 1998, and entitled “2-Alkylidene-19-Nor-Vitamin D Compounds” the specification of which is specifically incorporated herein by reference. The biological activity of 2MD is reported in U.S. patent application Ser. No. 09/616,164, filed Jul. 14, 2000, the specification of which is also specifically incorporated herein by reference.

2α-methyl-19-nor-(20S)-1α,25-dihydroxyvitamin D₃ (2AMD) is an analog of 1,25(OH)₂D₃ which has been shown to have in-vivo effects as a vitamin D receptor agonist at doses that do not elicit hypercalcemia. The overall synthesis of 2AMD is illustrated and described more completely in U.S. Pat. No. 5,945,410, issued Aug. 31, 1999; U.S. Pat. No. 6,127,559 issued Oct. 3, 2000; and U.S. Pat. No. 6,277,837 issued Aug. 21, 2001 which are incorporated herein by reference.

In one embodiment, the present invention therefore provides novel methods of preventing nephrotoxicity in a patient taking an immunosuppressive agent to prevent organ rejection by administering to the subject a therapeutically effective amount of 2-methylene-19-nor-(20S)-1α,25-dihydroxyvitamin D₃ (2MD) or pharmaceutically acceptable salts thereof, preferably without inducing hypercalcemia in the patient, where 2MD has the structure (I):

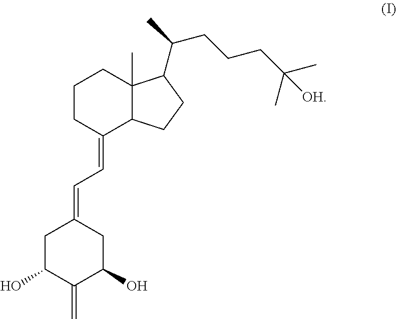 [SMILES: C=C1[C@H](O)CC(=C/C=C2\CCCC3(C)C2CCC3[C@@H](C)CCCC(C)(C)O)C[C@H]1O]

In one embodiment, the present invention therefore provides novel methods of preventing fibrosis in a subject by administering to the subject a therapeutically effective amount of 2MD or pharmaceutically acceptable salts thereof without inducing hypercalcemia in the subject, where 2MD has the structure (I).

In one embodiment, the present invention therefore provides novel methods of preventing nephrotoxicity in a patient taking an immunosuppressive agent to prevent organ rejection by administering to the subject a therapeutically effective amount of 2α-methyl-19-nor-(20S)-1α,25-dihydroxyvitamin D₃ (2AMD) or pharmaceutically acceptable salts thereof without inducing hypercalcemia in the patient, where 2AMD has the structure (II):

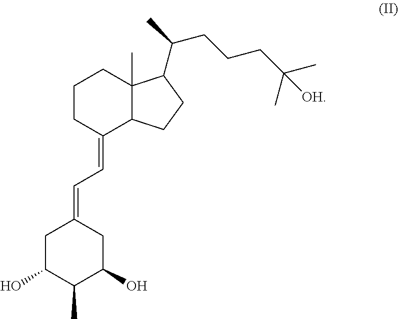

In one embodiment, the present invention therefore provides novel methods of preventing fibrosis in a patient by administering to the subject a therapeutically effective amount of 2AMD or pharmaceutically acceptable salts thereof without inducing hypercalcemia in the patient, where 2AMD has the structure (II).

By “pharmaceutically acceptable salt(s)” we mean those salts of compounds of the invention that are safe and effective for any number of administration methods and uses in mammals and that possess the desired biological activity.

By “hypercalcemia” we mean elevated calcium levels in the blood of more than 2 mg/100 ml above the normal. In a normal subject, calcium levels are approximately 9-10.5 mg/dL or 2.2-2.6 mmol/L. In cases of severe hypercalcemia (i.e., calcium levels above 15-16 mg/dL or 3.75-4 mmol/l) coma and cardiac arrest can develop. An increase in calcium of approximately 2 mg/100 ml is considered mild hypercalcemia. An increase in calcium levels of more than 2 mg/100 ml is considered severe hypercalcemia.

By “preventing” we mean a forestalling of a clinical symptom indicative of nephrotoxicity and/or fibrosis. Such forestalling includes, for example, the maintenance of normal organ functions and maintenance of normal organ tissue structures prior to the development of overt symptoms of nephrotoxic or fibrotic disease including, but not limited to, increased BUN, increased serum creatinine, urinary protein/creatinine ratio and collagen buildup in the tissue. Therefore, the term “preventing” includes the prophylactic treatment of subjects to guard them from the occurrence of nephrotoxicity and/or fibrosis. Preventing nephrotoxicity and/or fibrosis in a subject is also intended to include inhibiting or arresting the development of nephrotoxicity and/or fibrosis. Inhibiting or arresting the development of nephrotoxicity and/or fibrosis can include, for example, inhibiting or arresting the occurrence of collagen and other fibrotic proteins. Inhibiting or arresting the development of fibrosis also includes, for example, inhibiting or arresting the progression of one or more pathological conditions or chronic complications associated with fibrosis.

As used herein, “treat,” “treating” or “treatment” means amelioration, alleviation or ablation of a clinical symptom indicative of nephrotoxicity or fibrosis. Amelioration, alleviation or ablation of a clinical symptom includes, for example, arresting, preventing, reducing the severity of or slowing the progression of or causing the regression of a symptom of nephrotoxicity or fibrosis. For instance, lowering the amount of serum BUN, urinary protein/creatinine ratio or serum creatinine levels by at least 10% in response to treatment with 2MD or 2AMD. In a preferred embodiment, levels are reduced by 30%. In a most preferred embodiment, levels are reduced by 50%. Additionally, lowering the amount of tissue collagen as histologically determined by at least 10% in response to treatment with 2MD or 2AMD. In a preferred embodiment, levels are reduced by 30%. In a most preferred embodiment, levels are reduced by 50%.

Other pathological conditions, chronic complications or phenotypic manifestations of nephrotoxicity and/or fibrosis are known to those skilled in the art and can similarly be used as a measure of treating nephrotoxicity and/or fibrosis so long as there is a reduction in the severity of the condition, complication or manifestation associated with the disease.

By “subject” or “patient”, we mean mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” or “patient” does not denote a particular age or sex.

By “administering” we mean any means for introducing a compound into the body, preferably into the systemic circulation, as described in more detail below. Examples include but are not limited to oral, topical, buccal, sublingual, pulmonary, transdermal, transmucosal, as well as subcutaneous, intraperitoneal, intravenous, and intramuscular injection or in the form of liquid or solid doses via the alimentary canal.

By “therapeutically effective” we mean an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors. We dosed Sprague-Dawley rats with several dose levels of 2AMD or 2MD that would not cause significant hypercalcemia. We found that 5 ng/kg of 2AMD or 2MD per day is sufficient to prevent nephrotoxicity and/or fibrosis without dramatically increasing the subject's serum calcium levels. Furthermore, 2.5 ng/kg of 2AMD showed a reduction in collagen content while maintaining serum calcium levels within the physiologically normal range.

In one embodiment, the therapeutically effective amount ranges from between about 2.5-10 ng/kg/day. In an alternative embodiment, the therapeutically effective amount ranges from between about 3-9 ng/kg/day. In a preferred embodiment, the therapeutically effective amount is 5ng/kg/day. (See U.S. Pat. No. 5,843,928, which discloses suitable methods of administration and is hereby incorporated by reference herein). One would monitor the progression of nephrotoxicity and/or fibrosis by tracking symptoms, such as total urinary protein to creatinine ratio, creatinine, blood urea nitrogen (BUN), and tissue collagen content. One would expect these symptoms to slow in progression, or to improve.

Treatment with 2MD or 2AMD may continue through the course of treatment with an immunosuppressive agent. In one embodiment 2MD or 2AMD can be administered concurrently through the course of treatment of a CNI to the subject. In another embodiment, 2MD or 2AMD can be administered concurrently through the course of treatment of CsA to the subject. In another embodiment, 2MD or 2AMD can be administered daily throughout a subject's increased risk of developing fibrosis, such as, for example, throughout chronic Antibody-mediated rejection of a transplanted organ, the duration of a treatment with an immunosuppressant, a treatment with a chemotherapeutic agent, recovery from a surgical treatment, or acute injury. For some treatment would be life-long, whereas for others it would last until the fibrotic event has ended.

### Dosage and Administration

In one embodiment, the 2MD compound is the active pharmaceutical ingredient (API) of this invention. In one embodiment, the 2AMD compound is the active pharmaceutical ingredient of this invention. The API may be formulated in an oral pharmaceutical dosage form as a solution in innocuous solvents, emulsion, suspension or dispersion in suitable solvents or carriers. The API may also be formulated in various oral dosage forms, such as pills, tablets or capsules using suitable pharmaceutical solid carriers. Such pharmaceutical formulations may also contain other pharmaceutically suitable USP-approved inactive ingredients, excipients, such as stabilizers, anti-oxidants, binders, coloring agents, emulsifiers, and/or taste-modifying agents, which are referred to as USP approved inactive pharmaceutical ingredients.

The API may be administered orally, topically, parenterally or transdermally or by inhalation. The compound may be administered by injection or intravenous infusion using suitable sterile solutions. Topical dosage forms may be creams, ointments, patches, or similar vehicles suitable for transdermal and topical dosage forms.

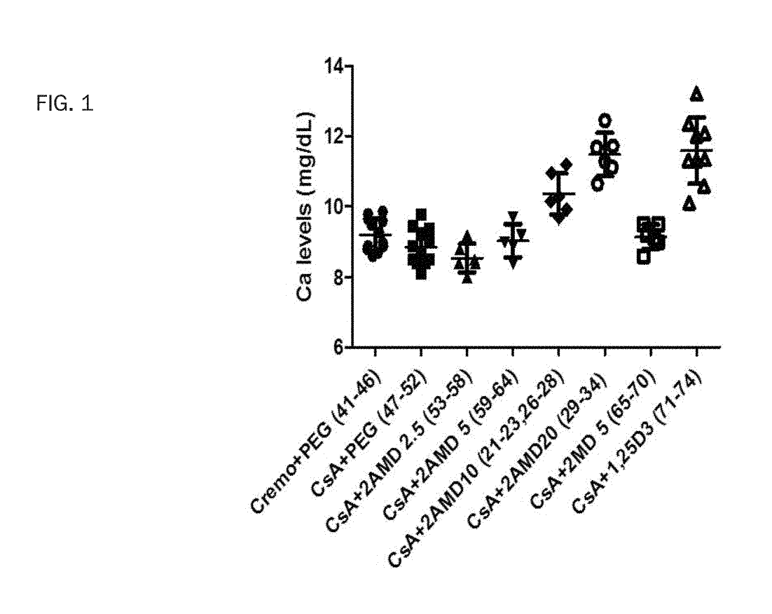

Doses in the range of 2.5-10 ng per day per kg bodyweight of the API may be used for the prevention or treatment of nephrotoxicity and/or fibrosis. All positive effects of 2MD and 2AMD are observed at dose levels that do not significantly raise serum calcium (FIG. 1). Such doses and dosing regimens may be adjusted to accommodate disease severity or progression, patient predisposition/at-risk/susceptible-to and other known criteria.

The API of this invention may also be a mixture or co-administration of two or more pharmaceutically active compounds. 2MD and 2AMD may also be administered in combination or co-administered with other pharmaceutically active ingratiates, or may be administered together in combination. In one embodiment 2MD or 2AMD is administered in combination with an immunosuppressive agent.

The pharmaceutically suitable oral carrier systems (also referred to as drug delivery systems, which are modern technology, distributed with or as a part of a drug product that allows for the uniform release or targeting of drugs to the body) preferably include FDA-approved and/or USP-approved inactive ingredients. Under 21 CFR 210.3(b)(8), an inactive ingredient is any component of a drug product other than the active ingredient. According to 21 CFR 210.3(b)(7), an active ingredient is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. As used herein, a kit (also referred to as a dosage form) is a packaged collection of related material.

As used herein, the oral dosage form includes capsules, a solid oral dosage form consisting of a shell and a filling, whereby the shell is composed of a single sealed enclosure, or two halves that fit together and which are sometimes sealed with a band, and whereby capsule shells may be made from gelatin, starch, or cellulose, or other suitable materials, may be soft or hard, and are filled with solid or liquid ingredients that can be poured or squeezed. The oral dosage form may also be a capsule or coated pellets, in which the drug is enclosed within either a hard or soft soluble container or “shell” made from a suitable form of gelatin. The drug itself may be in the form of granules to which varying amounts of coating have been applied or in a capsule coated extended release, in which the drug is enclosed within either a hard or soft soluble container or “shell” made from a suitable form of gelatin. Additionally, the capsule may be covered in a designated coating which releases a drug or drugs in such a manner to allow at least a reduction in dosing frequency as compared to that drug or drugs presented as a conventional dosage form.

The oral dosage form may further be a capsule delayed release, in which the drug is enclosed within either a hard or soft soluble container made from a suitable form of gelatin, and which releases a drug (or drugs) at a time other than promptly after administration, whereby enteric-coated articles are delayed release dosage forms. Capsule delayed release pellets, in which the drug is enclosed within either a hard or soft soluble container or “shell” made from a suitable form of gelatin are also useful. In these cases, the drug itself is in the form of granules to which enteric coating has been applied, thus delaying release of the drug until its passage into the intestines. Capsule extended release and capsule film-coated extended release are also useful.

Additionally, the capsule may be capsule film coated (which releases a drug or drugs in such a manner to allow at least a reduction in dosing frequency as compared to that drug or drugs presented as a conventional dosage form), capsule gelatin coated (a solid dosage form in which the drug is enclosed within either a hard or soft soluble container made from a suitable form of gelatin; through a banding process, the capsule is coated with additional layers of gelatin so as to form a complete seal), or capsule liquid filled (a solid dosage form in which the drug is enclosed within a soluble, gelatin shell which is plasticized by the addition of a polyol, such as sorbitol or glycerin, and is therefore of a somewhat thicker consistency than that of a hard shell capsule).

Typically, the active ingredients are dissolved or suspended in a liquid vehicle, granule (a small particle or grain), pellet (a small sterile solid mass consisting of a highly purified drug, with or without excipients, made by the formation of granules, or by compression and molding), or pellets coated extended release (a solid dosage form in which the drug itself is in the form of granules to which varying amounts of coating have been applied, and which releases a drug or drugs in such a manner to allow a reduction in dosing frequency as compared to that drug or drugs presented as a conventional dosage form).

Other forms include pills (a small, round solid dosage form containing a medicinal agent intended for oral administration), powder (an intimate mixture of dry, finely divided drugs and/or chemicals that may be intended for internal or external use), elixir (a clear, pleasantly flavored, sweetened hydroalcoholic liquid containing dissolved medicinal agents; it is intended for oral use), chewing gum (a sweetened and flavored insoluble plastic material of various shapes which when chewed, releases a drug substance into the oral cavity), syrup (an oral solution containing high concentrations of sucrose or other sugars; the term has also been used to include any other liquid dosage form prepared in a sweet and viscid vehicle, including oral suspensions), tablet (a solid dosage form containing medicinal substances with or without suitable diluents), tablet chewable (a solid dosage form containing medicinal substances with or without suitable diluents that is intended to be chewed, producing a pleasant tasting residue in the oral cavity that is easily swallowed and does not leave a bitter or unpleasant after-taste), tablet coated or tablet delayed release, tablet dispersible, tablet effervescent, tablet extended release, tablet film coated, or tablet film coated extended release where the tablet is formulated in such manner as to make the contained medicament available over an extended period of time following ingestion.

In other forms, a tablet for solution, tablet for suspension, tablet multilayer, tablet multilayer extended release may be provided, where the tablet is formulated in such manner as to allow at least a reduction in dosing frequency as compared to that drug presented as a conventional dosage form. A tablet orally disintegrating, tablet orally disintegrating delayed release, tablet soluble, tablet sugar coated, osmotic, and the like are also suitable.

The oral dosage form composition contains an active pharmaceutical ingredient and one or more inactive pharmaceutical ingredients such as diluents, solubilizers, alcohols, binders, controlled release polymers, enteric polymers, disintegrants, excipients, colorants, flavorants, sweeteners, antioxidants, preservatives, pigments, additives, fillers, suspension agents, surfactants (e.g., anionic, cationic, amphoteric and nonionic), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

As used herein, the injectable and infusion dosage forms include, but are not limited to, a liposomal injectable, which either consists of or forms liposomes (a lipid bilayer vesicle usually composed of phospholipids which is used to encapsulate an active drug substance), a parenteral injection (which includes a sterile preparation intended for parenteral use; five distinct classes of injections exist as defined by the USP are also suitable), or an emulsion injection (which includes an emulsion consisting of a sterile, pyrogen-free preparation intended to be administered parenterally or a lipid complex injection are also suitable).

Other forms include a powder for solution injection (which is a sterile preparation intended for reconstitution to form a solution for parenteral use); a powder for suspension injection (that is a sterile preparation intended for reconstitution to form a suspension for parenteral use); a powder lyophilized for liposomal suspension injection (which is a sterile freeze dried preparation intended for reconstitution for parenteral use which has been formulated in a manner that would allow liposomes (a lipid bilayer vesicle usually composed of phospholipids which is used to encapsulate an active drug substance, either within a lipid bilayer or in an aqueous space) to be formed upon reconstitution); or a powder lyophilized for solution injection (which is a dosage form intended for the solution prepared by lyophilization (“freeze drying”), a process which involves the removal of water from products in the frozen state at extremely low pressures).

It is intended for subsequent addition of liquid to create a solution that conforms in all respects to the requirements for injections; a powder lyophilized for suspension injection being a liquid preparation, intended for parenteral use that contains solids suspended in a suitable fluid medium and conforms in all respects to the requirements for Sterile Suspensions; the medicinal agents intended for the suspension are prepared by lyophilization (“freeze drying”), a process which involves the removal of water from products in the frozen state at extremely low pressures; a solution injection being a liquid preparation containing one or more drug substances dissolved in a suitable solvent or mixture of mutually miscible solvents that is suitable for injection; a solution concentrate injection being a sterile preparation for parenteral use which, upon the addition of suitable solvents, yields a solution conforming in all respects to the requirements for injections.

A suspension injection comprises a liquid preparation, suitable for injection, which consists of solid particles dispersed throughout a liquid phase in which the particles are not soluble that can also consist of an oil phase dispersed throughout an aqueous phase, or vice-versa. A suspension liposomal injection comprises a liquid preparation, suitable for injection, which consists of an oil phase dispersed throughout an aqueous phase in such a manner that liposomes (a lipid bilayer vesicle usually composed of phospholipids which is used to encapsulate an active drug substance, either within a lipid bilayer or in an aqueous space) are formed. A suspension sonicated injection comprises a liquid preparation, suitable for injection, which consists of solid particles dispersed throughout a liquid phase in which the particles are not soluble. In addition, the product is sonicated while a gas is bubbled through the suspension, and this results in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives. Formulations suitable for parenteral administration conveniently comprise a sterile oily or aqueous preparation of the active ingredient which is preferably isotonic with the blood of the recipient.

As used herein, inhalation dosage forms include, but are not limited to, aerosol being a product that is packaged under pressure and contains therapeutically active ingredients that are released upon activation of an appropriate valve system intended for topical application to the skin as well as local application into the nose (nasal aerosols), mouth (lingual and sublingual aerosols), or lungs (inhalation aerosols); foam aerosol being a dosage form containing one or more active ingredients, surfactants, aqueous or nonaqueous liquids, and the propellants, whereby if the propellant is in the internal (discontinuous) phase (i.e., of the oil-in-water type), a stable foam is discharged, and if the propellant is in the external (continuous) phase (i.e., of the water-in-oil type), a spray or a quick-breaking foam is discharged; metered aerosol being a pressurized dosage form consisting of metered dose valves which allow for the delivery of a uniform quantity of spray upon each activation; powder aerosol being a product that is packaged under pressure and contains therapeutically active ingredients, in the form of a powder, that are released upon activation of an appropriate valve system; and, aerosol spray being an aerosol product which utilizes a compressed gas as the propellant to provide the force necessary to expel the product as a wet spray and being applicable to solutions of medicinal agents in aqueous solvents.

As used herein, transdermal dosage form includes, but is not limited to, a patch being a drug delivery system that often contains an adhesive backing that is usually applied to an external site on the body, whereby the ingredients either passively diffuse from, or are actively transported from, some portion of the patch, and whereby depending upon the patch, the ingredients are either delivered to the outer surface of the body or into the body; and, other various types of transdermal patches such as matrix, reservoir and others known in the art.

As used herein, the topical dosage form includes various dosage forms known in the art such as lotions (an emulsion, liquid dosage form, whereby this dosage form is generally for external application to the skin), lotion augmented (a lotion dosage form that enhances drug delivery, whereby augmentation does not refer to the strength of the drug in the dosage form), gels (a semisolid dosage form that contains a gelling agent to provide stiffness to a solution or a colloidal dispersion, whereby the gel may contain suspended particles) and ointments (a semisolid dosage form, usually containing <20% water and volatiles and >50% hydrocarbons, waxes, or polyols as the vehicle, whereby this dosage form is generally for external application to the skin or mucous membranes).

Ointment augmented (an ointment dosage form that enhances drug delivery, whereby augmentation does not refer to the strength of the drug in the dosage form), creams (an emulsion, semisolid dosage form, usually containing >20% water and volatiles and/or <50% hydrocarbons, waxes, or polyols may also be used as the vehicle, whereby this dosage form is generally for external application to the skin or mucous membranes), cream augmented (a cream dosage form that enhances drug delivery, whereby augmentation does not refer to the strength of the drug in the dosage form), emulsions (a dosage form consisting of a two-phase system comprised of at least two immiscible liquids, one of which is dispersed as droplets, internal or dispersed phase, within the other liquid, external or continuous phase, generally stabilized with one or more emulsifying agents, whereby emulsion is used as a dosage form term unless a more specific term is applicable, e.g. cream, lotion, ointment), suspensions (a liquid dosage form that contains solid particles dispersed in a liquid vehicle), suspension extended release, pastes (a semisolid dosage form, containing a large proportion, 20-50%, of solids finely dispersed in a fatty vehicle, whereby this dosage form is generally for external application to the skin or mucous membranes), solutions (a clear, homogeneous liquid dosage form that contains one or more chemical substances dissolved in a solvent or mixture of mutually miscible solvents), and powders are also suitable.

Jellies (a class of gels, which are semisolid systems that consist of suspensions made up of either small inorganic particles or large organic molecules interpenetrated by a liquid—in which the structural coherent matrix contains a high portion of liquid, usually water) and films (a thin layer or coating), including film extended release (a drug delivery system in the form of a film that releases the drug over an extended period in such a way as to maintain constant drug levels in the blood or target tissue) and film soluble (a thin layer or coating which is susceptible to being dissolved when in contact with a liquid) are also suitable.

Sponges (a porous, interlacing, absorbent material that contains a drug, whereby it is typically used for applying or introducing medication, or for cleansing, and whereby a sponge usually retains its shape), and swabs (a small piece of relatively flat absorbent material that contains a drug, whereby a swab may also be attached to one end of a small stick, and whereby a swab is typically used for applying medication or for cleansing) are also suitable.

Patches (a drug delivery system that often contains an adhesive backing that is usually applied to an external site on the body, whereby its ingredients either passively diffuse from, or are actively transported from, some portion of the patch, whereby depending upon the patch, the ingredients are either delivered to the outer surface of the body or into the body, and whereby a patch is sometimes synonymous with the terms ‘extended release film’ and ‘system’), patch extended release (a drug delivery system in the form of a patch that releases the drug in such a manner that a reduction in dosing frequency compared to that drug presented as a conventional dosage form, e.g., a solution or a prompt drug-releasing, conventional solid dosage form), patch extended release electronically controlled (a drug delivery system in the form of a patch which is controlled by an electric current that releases the drug in such a manner that a reduction in dosing frequency compared to that drug presented as a conventional dosage form, e.g., a solution or a prompt drug-releasing, conventional solid dosage form), and the like are also suitable. The various topical dosage forms may also be formulated as immediate release, controlled release, sustained release, or the like.

The topical dosage form composition contains an active pharmaceutical ingredient and one or more inactive pharmaceutical ingredients such as excipients, colorants, pigments, additives, fillers, emollients, surfactants (e.g., anionic, cationic, amphoteric and nonionic), penetration enhancers (e.g., alcohols, fatty alcohols, fatty acids, fatty acid esters and polyols), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description of the novel methods of the present invention are to be regarded as illustrative in nature and not restrictive.

### EXAMPLES

### Example 1

### Treatment of Nephrotoxicity and Fibrosis Caused by CsA with 2AMD and 2MD

Vitamin D analogs have been used in multiple clinical trials involving chronic kidney disease, hemodialysis, and hyper-parathyroidism. Ongoing trials testing the effects of vitamin D supplementation on patients undergoing kidney transplantation utilize cholecalciferol [1], which is known to have hypercalcemic effects with deleterious outcomes on patients' cardiovascular systems.

Novel selective analogs of vitamin D, such as 2-methylene-19-nor-(20S)-1α,25-dihydroxivitamin D3 (2MD) are known to be hyperactive Vitamin D receptor agonists at doses that do not elicit hypercalcemia [2, 3]. Another such analog, 2AMD (2a-methyl-19-nor-(20S)-1α,25-dihydroxyvitaminD3) is studied herein for its ability to protect rat kidneys from CsA-induced nephrotoxicity. This model is relevant to transplantation as CsA is an immunosuppressant given to reduce organ rejection but which eventually promotes renal vasoconstriction ending in epithelial cell apoptosis and interstitial fibrosis [4]. Various doses of 2AMD are tested to identify the nephroprotective dose which would not raise serum calcium levels and it is found that 2AMD at 5 ng/kg rat body weight was optimal in nephroprotection.

### Methods

### Animals

Sprague-Dawley male rats (Harlan), weighing approximately 250 g were started on a low sodium diet (TD94268, 0.1% NaCl) one week prior to start of CsA treatment. Rats were randomly assigned to groups of 6 each. Each animal received daily injections under 2% isofluorane. CsA (Sandimmune, Novartis) in Cremophor EL, 33% EtOH was given at 20 mg/kg/day. Vitamin D analogs were dissolved in polyethylene glycol, 0.5% ethanol (PEG). Table 1 shows the layout of two combined cohorts, one injected intraperitoneal and the second, subcutaneous. All rats were provided with food and water ad-libitum as per standard procedure. Animals were dosed daily for 28 days with the last dose given approximately 24 h prior to harvest (except for 5 animals receiving 1,25(OH)₂D3 which were dosed 48 h prior to harvest). Animals were placed under anesthesia, urine and blood (aorta) were collected and kidneys removed; rats were euthanized by pneumothorax puncture. Kidneys were sectioned longitudinally with one half placed into 10% formalin and the other half snap frozen into liquid nitrogen. Blood was allowed to clot for about 1 h followed by spinning and removing of serum. Tissues in formalin were embedded in paraffin and sectioned for further study. Serum, urine and frozen kidney tissues were stored at −80° C.

TABLE 1
Vitamin D analog study design, two cohorts combined

| content | dose ng/kg body weight | n | cohort |
|---|---|---|---|
| Cremo + PEG | — | 12 | 1, 2 |
| CsA + PEG | — | 12 | 1, 2 |
| CsA + 2AMD | 2.5 | 6 | 2 |
| CsA + 2AMD | 5 | 5* | 2 |
| CsA + 2AMD | 10 | 6 | 1 |
| CsA + 2AMD | 20 | 6 | 1 |
| CsA + 2MD | 5 | 6 | 2 |
| CsA + 1,25 D3 | 100 | 9** | 1, 2 |
| saline | | 2 | 1 |

*rat died mid-study, necropsy showed disseminated intravascular coagulation from sepsis.
**rat died of unknown cause, no necropsy

### Serum and Urine Tests

Serum was analyzed for BUN, creatinine and calcium levels. The BUN and creatinine measurements were performed using IDEXX cartridges measured on a VETTEST Chemistry Analyzer. Calcium levels were measured in 0.1% lanthanum chloride by atomic absorption spectroscopy (Perkin Elmer Model 3110).

Urine testing to determine protein/creatinine ratio was performed using IDEXX cartridges; protein levels were also measured using BCA protein determination.

### Histochemistry

Kidneys fixed in formalin were embedded in paraffin, sectioned and stained with Hematoxilin/eosin and with pricrosirius red, which stains mostly collagens I and III. Stained sections were imaged on an upright brightfield Nikon microscope with polarizer capabilities to assess the birefringence of the picrosirius -stained collagen fibers. Quantization of staining was carried out using Image J from 5 images obtained at random within the central cortical area and excluding blood vessels. For H&E, the eosin staining (pink) prevalent in native tissue was quantified

### Statistics

Data was analyzed using Graphpad Prism software with the standard student t-test or One-way Anova operations.

### Results

The aim of the study was to ascertain the effects of 2AMD, a vitamin D analog similar to 2MD which functions without raising serum calcium levels thus avoiding the deleterious effects associated with hypercalcemia [5].

Serum calcium levels were obtained from blood collected approximately 24 h following the last vitamin D analog dose. FIG. 1 shows the calcium level for rats treated with 1,25(OH)₂ D3 was increased by about 25% with respect to rats with vehicles or CsA+PEG. Rats treated with CsA and either 2AMD or 2MD at 5 ng/kg rat body weight presented an increase of about 2% compared to the vehicle or CsA+PEG group. Thus 2AMD and 2MD performed as expected in terms of producing a negligible rise in serum calcium. Serum calcium levels were highly dose-dependent with an evident linear relationship with dose of 2AMD.

Several parameters were used to test for protective effects by 2AMD on nephrotoxicity induced by Cyclosporin treatment, such as weight change, serum BUN and creatinine, urinary protein to creatinine ratio (UPC), histological changes and increase in fibrosis (as determined histologically by levels of collagen stained by pricrosirius red). Not all parameters have been measured for both cohorts.

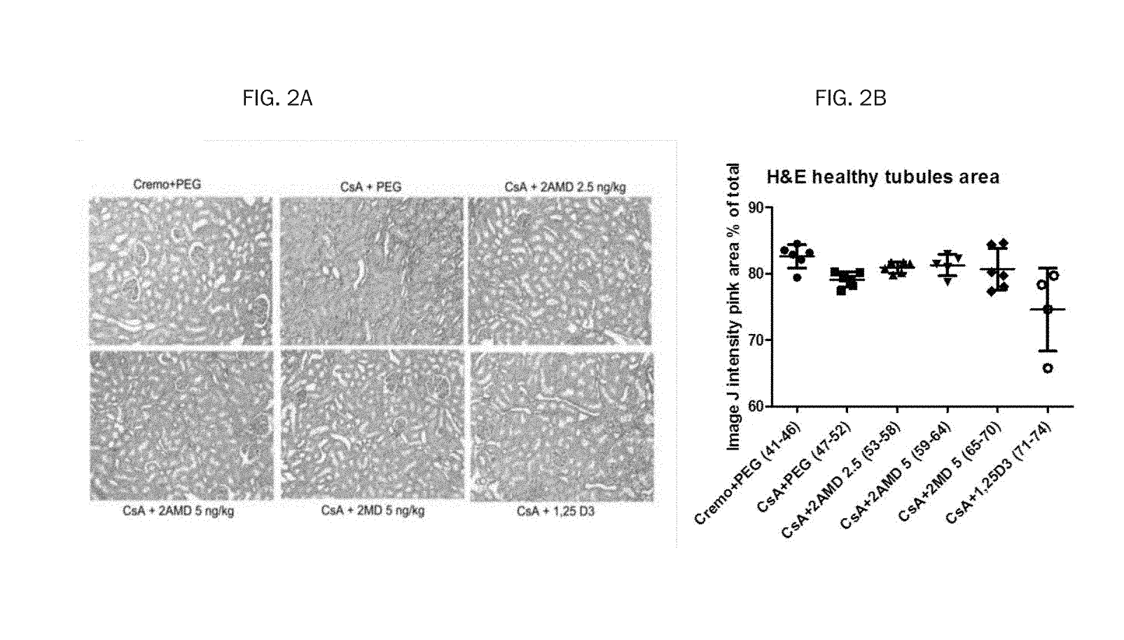

Histological examination of H&E stained kidney sections revealed some tubular condensation and striped fibrosis with CsA treatment. Representative images from the vehicle, CsA+PEG and CsA+2AMD at 2.5 and 5 ng/kg, 2MD at 5 ng/kg, or 1,25(OH)₂D3 are shown in FIG. 2. Eosinophilic staining is prevalent in normal tubules and was lower in images from kidneys from rats treated with CsA. This was determined by histological analysis of the kidneys by quantitation of eosin (pink as vs purple) in H&E-stained tissues. 2AMD at both 2.5 and 5 ng/kg promoted an increase in eosinophilic tissue compared to the CsA treated group.

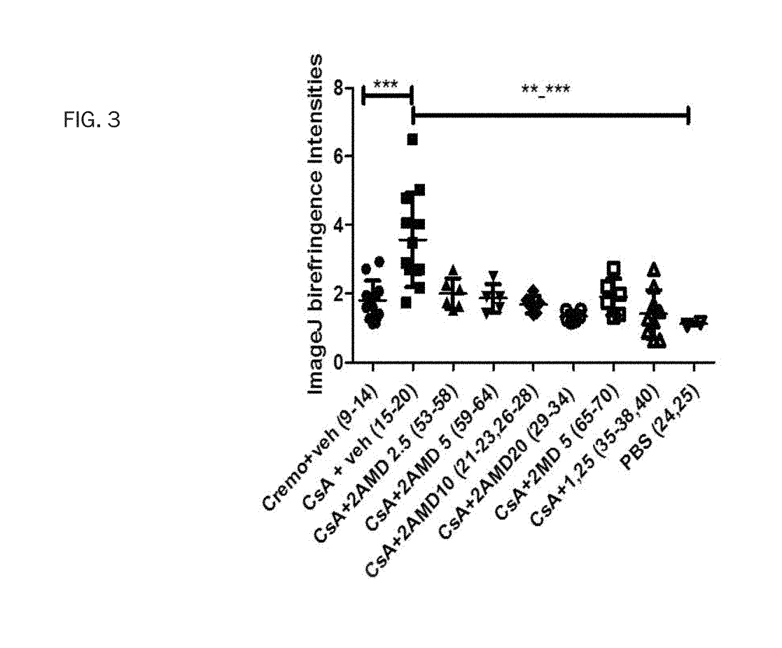

Picrosirius red staining of kidney tissue sections outlines collagens I and III which are increased in fibrotic tissue. Quantification of the birefringence of collagen fibers under polarized light indicated a significant increase in the CsA treated group compared to the vehicle group. 2AMD at 5 ng/kg decreased the CsA-promoted effect by about 30%, as shown in FIG. 3. Similar effects were observed for all other groups treated with vitamin D analogs.

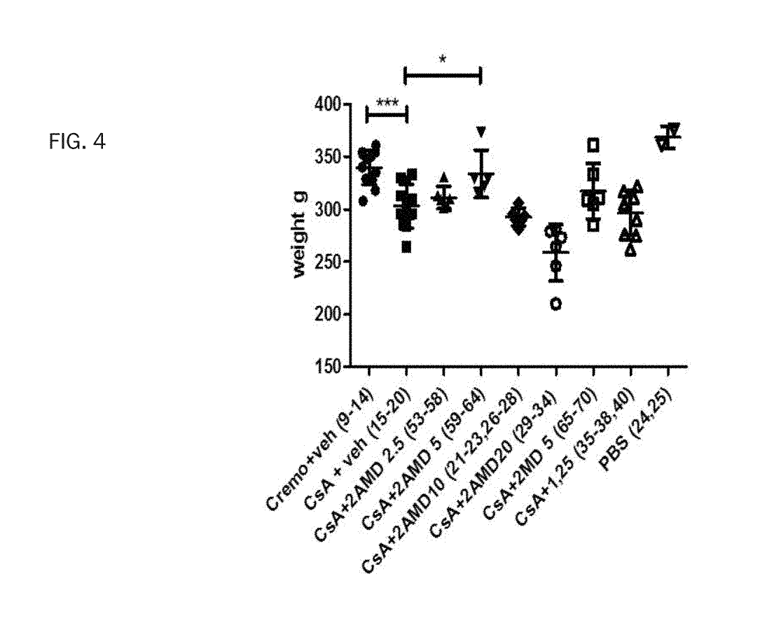

Shown in FIG. 4 is the change in weight obtained on the day of harvest as a percent of the weight of each rat on the day CsA and PEG or vitamin D analog injections were started. As expected, rats treated with CsA+PEG weighed about 30 g less than the vehicle group. Most of the groups treated with CsA and vitamin D analogs decreased in weight similarly to the CsA+PEG group, except for the group treated with 2AMD at 5 ng/kg which caused the smallest change in weight due to CsA, dropping only by a mean of 5 g from the rats' original weights.

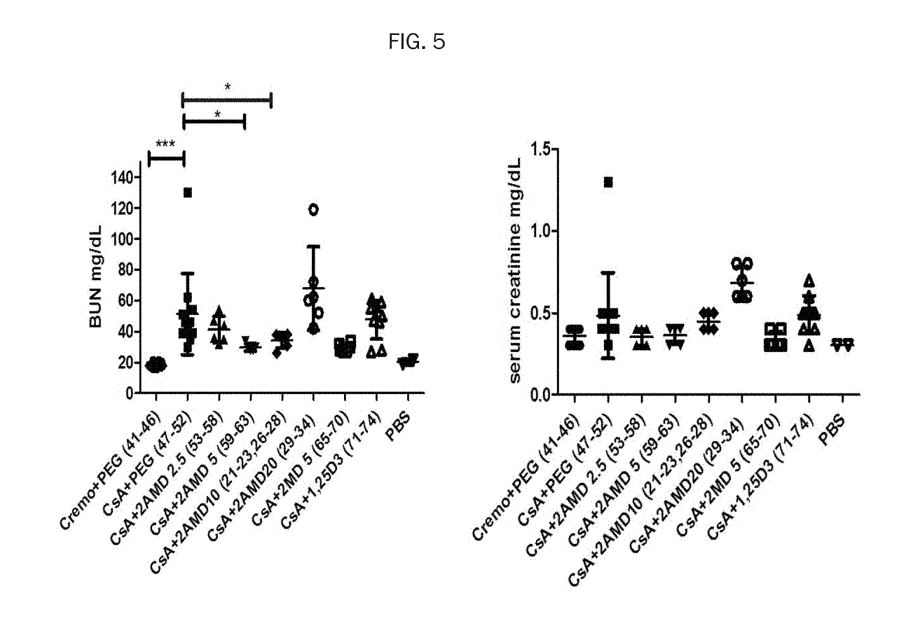

Other tests included serum blood urea nitrogen (BUN), which is a measure of kidney function. Normal BUN is the range of 9-21 mg/dL. All rats had higher than normal BUN but CsA-induced nephrotoxicity was demonstrated by a significant increase in serum BUN compared to rats treated with vehicle (shown in FIG. 5). Rats treated with 2AMD at both 5 and 10 ng/kg and with 2MD at 5 ng/kg significantly decreased BUN by about 30% compared to the CsA-treated group. Normal serum creatinine is below 0.6 mg/dL and was normal in most of the treatment groups except for 2AMD at 20 ng/kg (FIG. 5).

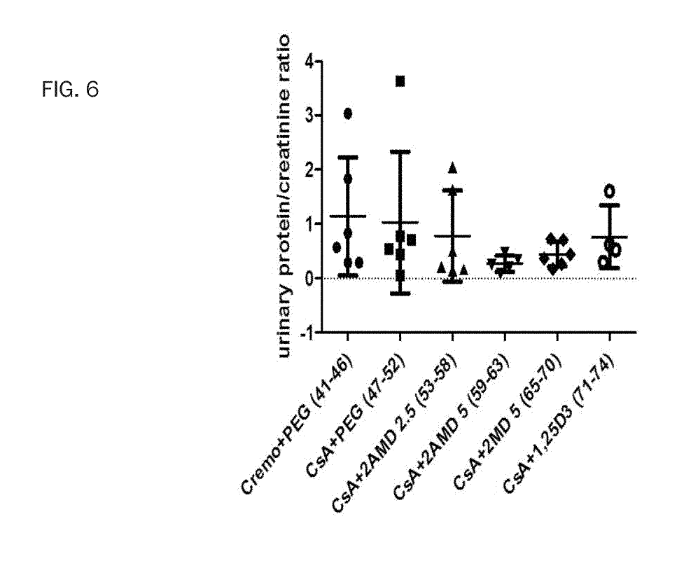

Another clinically important measure of kidney function is the urinary protein/creatinine ratio (UPC). There was no clear increase in UPC with CsA treatment in that the vehicle-treated rats presented similar values to the CsA treated group. However, the mean UPC values for both 2AMD and 2MD at 5 ng/kg were about 74% lower than the mean UPC for the CsA group (FIG. 6). The reason for the variance and high UPC in the control group is unclear, but it is apparent that both analogs at 5 ng/kg counteract it. One explanation for the variance in the control groups may be that urine measurements were obtained from a spot urine collection (at harvest), whereas more accurate values could be obtained from a 24 h urine collection.

For some of the measured parameters, there is an abnormal result produced by 2AMD at 20 ng/kg, which is 4× the level found to be most therapeutic in terms of nephrotoxicity. For several of those parameters, such as serum calcium there is a linear relationship with dose, suggesting that tight control of effect is possible by adjusting dosage of 2AMD. Table 2 summarizes the values obtained for 2AMD in comparison to 2MD, both at the most effective dose of 5 ng/kg. Both groups were part of the same cohort.

TABLE 2
Test values comparing 2AMD and 2MD at 5 ng/kg.

| | | | 5 ng/kg | |
|---|---|---|---|---|
| | Cremo + | CsA + | CsA + | CsA + |
| | PEG | PEG | 2AMD | 2MD |
| Serum Ca, mg/dL | 9 | 8.7 | 9 | 9.1 |
| weight, g | 339.8 | 305 | 333.8 | 317.3 |
| BUN, mg/dL | 16 | 51 | 28 | 27 |
| Serum creatinine, mg/dL | 0.36 | 0.48 | 0.36 | 0.33 |
| Urinary protein/creatinine ratio | 1.1 | 1 | 0.2 | 0.4 |
| Picrosirius staining (collagens) | 1.6 | 3.6 | 1.8 | 1.7 |
| number of rats | 12 | 12 | 5 | 6 |

### Discussion and Conclusions

We show CsA induced statistically significant changes compared to vehicle in rat weight, serum BUN, serum creatinine, collagen and H&E. Compared to CsA-treated rats, 2AMD at 5 ng/kg significantly reduced BUN and collagen and was associated with increased eosin (healthy) staining by H&E. 2AMD also decreased the urine/protein ratio as compared to both vehicle and CsA controls. Similar to 2MD, 2AMD up to 5 ng/kg did not increase serum calcium levels compared to vehicle or CsA only treated rats. All doses of 2AMD were effective at reducing fibrosis, and 2AMD at 10 ng/kg also decreased BUN, however that dose produced a 12% rise in calcium. It appears that serum calcium levels are highly controllable by dose of 2AMD.

As mentioned, clinical trials for renal transplants have tested calcitriol or paricalcitol to counteract vitamin D insufficiency [1] or secondary hyperparathyroidism [6]. We show improvement in both renal function tests and fibrosis with 2AMD. This analog should be a good candidate for clinical trials in kidney transplantation with outcomes assessed as fibrotic index, graft and patient survival. Indeed, the observation that all Vitamin D analogs tested diminished collagen would suggest possible indications for vitamin D in other fibrotic diseases, which are responsible for 45% of deaths world-wide [7]. In addition to graft failure, fibrotic diseases include idiopathic pulmonary fibrosis, liver fibrosis, diabetic nephropathy and scleroderma.

### Example 2

### Treatment of Fibrosis

The unilateral ureteral obstruction (UUO) model is a surgical model representing a rodent equivalent of acute kidney injury [1-3]. Obstruction results in marked dilatation of the ureter together with reduced renal blood flow and glomerular filtration. Renal histology demonstrates tubular atrophy and increasingly severe interstitial renal inflammation and fibrosis.

Male Sprague-Dawley rats weighing approximately 200-350 g were acclimated and fed ad libitum a standard diet (Teklad 8604 or 2018, Harlan, Madison Wis.). Briefly, under 2% isoflurane anesthesia, the left kidney and ureter were exposed through a midline or a flank incision. The ureter was ligated using 5-0 or 6-0 braided silk suture material. The ligated ureter and kidney were returned to the abdominal cavity and the incision was closed in two layers with interrupted sutures and VETBOND tissue adhesive or staples. The right or contralateral kidney was used as a control. Sham operated animals were treated as the UUO animals except their ureter was not ligated (not shown).

Animals were given buprenorphine (0.05 mg/kg) prior to returning them to their cages where they were kept on standard water and chow until sacrifice at the designated times. Animals were euthanized under CO₂, following a midline incision blood was collected via cardiac puncture and the kidneys were removed. The midsection of each kidney was placed in 10% formalin for paraffin embedding and histological staining. The bottom sections of each kidney were snap frozen and kept at -80° C.

2AMD dissolved in vehicle or vehicle (Neobee M-5 oil) was administered orally for a total of 14 days starting on the day of surgery. For a set of rats, 2AMD was given 7 days prior to surgery and continued up to 7 days post-surgery (−7+7) when rats were euthanized. Vehicle-receiving rats were given a volume of oil equivalent to that given for 2AMD 1× (2.5 ng/kg) or 2× (5 ng/kg) in cohorts depicted below.

TABLE 3
2AMD fibrosis study cohorts

| duration, | admin. | | dose ng/kg | | cohort |
|---|---|---|---|---|---|
| days | mode | content | body weight | n | ID# |
| −7 + 7 | oral | vehicle 1X | — | 2 | 6 |
| −7 + 7 | oral | vehicle 2X | — | 6 | 3 |
| −7 + 7 | oral | 2AMD 1X | 2.5 | 2 | 6 |
| −7 + 7 | oral | 2AMD 2X | 5 | 9 | 2, 4, 6 |
| 14 | oral | vehicle 1X | — | 3 | 5 |
| 14 | oral | vehicle 2X | — | 4 | 1, 5 |
| 14 | oral | 2AMD 1X | 2.5 | 3 | 5 |
| 14 | oral | 2AMD 2X | 5 | 6 | 4, 5 |

### Results

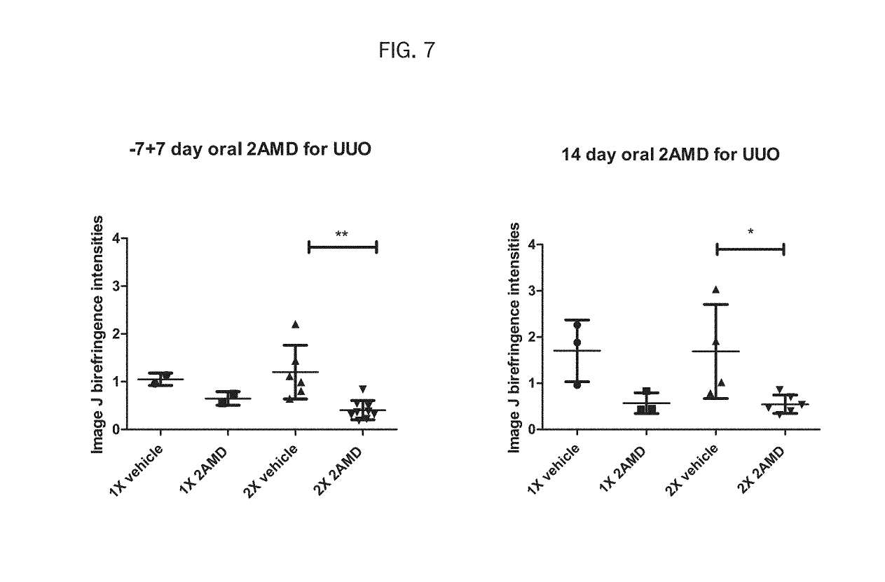

Shown in FIG. 7 is the quantization of picrosirius staining, representing collagen content associated with fibrosis. As expected, total collagen as detected for vehicles was higher after 14 days of UUO (right panel) than after 7 days (left). However, for both experimental setups, either giving 2AMD 7 days prior to UUO or administering on the day of UUO, there was a significant (3-fold) decrease in collagen content with 2AMD at 5 ng/kg, respectively after 7 or 14 days following UUO.

### Discussion and Conclusion

We have shown in an acute renal injury model (UUO) that 2AMD at 5 ng/kg/d is statistically effective in reducing fibrosis as judged by collagen deposition. These results indicate that 2AMD will be effective in not only in renal injury but also in other fibrotic diseases involving other organs including but not limited to idiopathic pulmonary fibrosis, COPD, cystic fibrosis, liver fibrosis, cirrhosis, diabetic nephropathy, renal fibrosis , nephrogenic system fibrosis, atrial fibrosis, endomyocardial fibrosis, cardiovascular remodeling, arthrofibrosis, myelofibrosis, Chron's disease, scleroderma, idiopathic retroperitoneal fibrosis, pancreatic fibrosis, IgG4-related disease, graft versus host disease, rejection of transplanted organ, glaucoma, post-traumatic or post-burn fibrotic changes such as scar formation which restrict movements or cause cosmetic problems, and some cancers, including but not limited to breast and pancreatic.

### REFERENCES

1. Courbebaisse, M., et al., VITamin D supplementation in renAL transplant recipients (VITALE): a prospective, multicentre, double-blind, randomized trial of vitamin D estimating the benefit and safety of vitamin D3 treatment at a dose of 100,000 UI compared with a dose of 12,000 UI in renal transplant recipients: study protocol for a double-blind, randomized, controlled trial. Trials, 2014. 15: p. 430.
2. Deluca, H. F., The development of a bone-and parathyroid-specific analog of vitamin D: 2-methylene-19-Nor-(20S)-lalpha,25-dihydroxyvitamin D3. Bonekey Rep, 2014. 3: p. 514.
3. Zella, J. B., et al., Novel, selective vitamin D analog suppresses parathyroid hormone in uremic animals and postmenopausal women. Am J Nephrol, 2014. 39(6): p. 476-83.
4. Burdmann, E. A., et al., Cyclosporine nephrotoxicity. Semin Nephrol, 2003. 23(5): p. 465-76.
5. Plum, L. A. and H. F. DeLuca, Vitamin D, disease and therapeutic opportunities. Nat Rev Drug Discov, 2010. 9(12): p. 941-55.
6. Trillini, M., et al., Paricalcitol for secondary hyperparathyroidism in renal transplantation. J Am Soc Nephrol, 2015. 26(5): p. 1205-14.
7. Nanthakumar, C. B., et al., Dissecting fibrosis: therapeutic insights from the small-molecule toolbox. Nat Rev Drug Discov, 2015. 14(10): p. 693-720.
8. Chevalier, R. L., M. S. Forbes, and B. A. Thornhill, Ureteral obstruction as a model of renal interstitial fibrosis and obstructive nephropathy. Kidney Int, 2009. 75(11): p. 1145-52.
9. Forbes, M. S., et al., Fight-or-flight: murine unilateral ureteral obstruction causes extensive proximal tubular degeneration, collecting duct dilatation, and minimal fibrosis. Am J Physiol Renal Physiol, 2012. 303(1): p. F120-9.
10. Ucero, A. C., et al., Unilateral ureteral obstruction: beyond obstruction. Int Urol Nephrol, 2014. 46(4): p. 765-76.

We claim:

1. A method of treating fibrosis or symptoms thereof in a patient, the method comprising administering a therapeutically effective amount of a compound selected from the group consisting of 2-methylene-19-nor-(20S)-1α,25-(OH)₂D₃ (2MD) and 2α-methyl-19-nor-(20S)-1α,25-(OH)₂D₃ (2AMD), or pharmaceutically acceptable salts thereof, to the patient.

2. The method of claim 1 wherein hypercalcemia is not induced in the patient during treatment.

3. The method of claim 1 wherein the compound is formulated in a formulation selected from the group consisting of an oral, topical, transdermal, parenteral, injection, and infusion dosage form.

4. The method of claim 1 wherein the fibrosis is a result of treatment with a calcineurin inhibitor (CNI).

5. The method of claim 4 wherein the CNI is cyclosporine (CsA).

6. The method of claim 1 wherein the fibrosis is a result of acute renal injury.

7. The method of claim 1 wherein the compound is 2MD.

8. The method of claim 1 wherein the compound is 2AMD.

9. The method of claim 1 wherein the patient is a mammal.

10. The method of claim 8 wherein the mammal is a human.

11. The method of claim 1 wherein the therapeutically effective amount of the compound is in the range of 2.5-10 ng/kg/day.

12. The method of claim 10 wherein the therapeutically effective amount of the compound is in the range of 3-9 ng/kg/day.

13. The method of claim 11 wherein the therapeutically effective amount of the compound is 5 ng/kg/day.

14. The method of claim 1 wherein the compound is administered by injection.

15. The method of claim 13 wherein the compound is administered by intraperitoneal injection.

16. The method of claim 13 wherein the compound is administered by subcutaneous injection.

17. The method of claim 1 wherein the compound is administered orally.

18. The method of claim 1 wherein the compound is administered in combination with a CNI.

19. The method of claim 17 wherein the CNI is CsA.

20. The method of claim 1 wherein the fibrosis is fibrosis of the kidney.

21. The method of claim 1 wherein the fibrosis is fibrosis of the lung.

22. A method of treating nephrotoxicity or symptoms thereof in a patient, the method comprising administering a therapeutically effective amount of a compound selected from the group consisting of 2-methylene-19-nor-(20S)-1α,25-(OH)₂D₃ (2MD) and 2α-methyl-19-nor-(20S)-1α,25-(OH)₂D₃ (2AMD) or pharmaceutically acceptable salts thereof to the patient.

23. The method of claim 21 wherein hypercalcemia is not induced in the patient during treatment.

24. The method of claim 21 wherein the compound is formulated in a formulation selected from the group consisting of an oral, topical, transdermal, parenteral, injection, and infusion dosage form.

25. The method of claim 21 wherein the nephrotoxicity is a result of treatment with a calcineurin inhibitor (CNI).

26. The method of claim 24 wherein the CNI is cyclosporine (CsA).

27. The method of claim 21 wherein the compound is 2MD.

28. The method of claim 21 wherein the compound is 2AMD.

29. The method of claim 21 wherein the patient is a mammal.

30. The method of claim 28 wherein the mammal is a human.

31. The method of claim 21 wherein the therapeutically effective amount of the compound is in the range of 2.5-10 ng/kg/day.

32. The method of claim 30 wherein the therapeutically effective amount of the compound is in the range of 3-9 ng/kg/day.

33. The method of claim 31 wherein the therapeutically effective amount of the compound is 5 ng/kg/day.

34. The method of claim 21 wherein the compound is administered by injection.

35. The method of claim 33 wherein the compound is administered by intraperitoneal injection.

36. The method of claim 33 wherein the compound is administered by subcutaneous injection.

37. The method of claim 21 wherein the compound is administered orally.

38. The method of claim 21 wherein the compound is administered in combination with a CNI.

39. The method of claim 37 wherein the CNI is CsA.

40. The method of claim 21 wherein the patient has or will undergo an organ transplantation procedure.

41. A composition of matter comprising a compound selected from the group consisting of 2-methylene-19-nor-(20S)-1α,25-(OH)₂D₃ (2MD) and 2α-methyl-19-nor-(20S)-1α,25-(OH)₂D₃ (2AMD), or pharmaceutically acceptable salts thereof, and an immunosuppressive agent.

42. The composition of claim 40 in which the compound is 2MD.

43. The composition of claim 40 in which the compound is 2AMD.

44. The composition of claim 40 in which the immunosuppressive agent is a CNI.

45. The composition of claim 43 in which the CNI is CsA.
